(19) United States
(12) Patent Application Publication
(10) Pub. No.: US 2026/0049091 A1
(43) Pub. Date: Feb. 19, 2026

(54) 2-(1,8-DIETHYL-1,3,4,9-TETRAHYDROPYRANO[3,4-B]INDOL-1-YL)-1-(4-METHYLPIPERAZIN-1-YL)ETHAN-1-ONE ETODOLAC DERIVATIVE AS EUKARYOTIC ELONGATION FACTOR 2 KINASE (EEF2K) INHIBITOR FOR TARGETED CANCER THERAPY

(71) Applicant: BAHCESEHIR UNIVERSITESI, Istanbul (TR)

(72) Inventors: Ferah COMERT ONDER, Canakkale (TR); Bulent OZPOLAT, Houston, TX (US); Serdar DURDAGI, Istanbul (TR); Mehmet AY, Canakkale (TR); Pinar SIYAH, Istanbul (TR)

(73) Assignee: BAHCESEHIR UNIVERSITESI, Istanbul (TR)

(21) Appl. No.: 18/995,984

(22) PCT Filed: Mar. 29, 2024
(86) PCT No.: PCT/TR2024/050321

§ 371 Date: Jan. 17, 2025

(30) Foreign Application Priority Data
Jun. 1, 2023 (TR) 2023/006391

(51) Int. Cl. C07D 491/052

(52) U.S. Cl. CPC C07D 491/052

(57) ABSTRACT

A compound derivative is provided, where R in the compound of formula A is 4-methylpiperazine, and the compound derivative can be used in the treatment of cancer and other diseases through the development of small molecules as eukaryotic elongation factor 2 kinase (eEF2K) enzyme inhibitors that are active in breast, pancreatic, brain, ovarian, lung, skin and blood cancers.

### CROSS REFERENCE TO THE RELATED APPLICATIONS

This application is the national phase entry of International Application No. PCT/TR2024/050321, filed on Mar. 29, 2024, which is based upon and claims priority to Turkish Patent Application No. 2023/006391, filed on Jun. 1, 2023, the entire contents of which are incorporated herein by reference.

### TECHNICAL FIELD

The invention relates to a compound derivative that can be used in the treatment of cancer and other diseases through the development of small molecules as eukaryotic elongation factor 2 kinase (eEF2K) enzyme inhibitors that are active in breast, pancreatic, brain, ovarian, lung, skin and blood cancers.

### BACKGROUND

Eukaryotic elongation factor 2 kinase (eEF2K), also known as calmodulin-dependent protein kinase III (CAMKIII), is a member of the atypical alpha kinase family and was initially shown to have a role in the regulation of protein synthesis through eukaryotic elongation factor 2 (eEF2) phosphorylation. [1-3]
eEF2K has been shown to promote cell proliferation, survival, cell migration and invasion, and tumor growth in highly aggressive solid tumors, including triple negative breast cancer (TNBC), pancreatic, lung and ovarian cancers. [4-10]

Previous studies have reported that eEF2K is highly upregulated in TNBC cells and that this is associated with metastatic disease and shorter patient survival in TNBC, lung cancer and ovarian cancer. [4-11]

Furthermore, using various genetic silencing technologies, in vivo inhibition of eEF2K has been reported to suppress tumor growth in TNBC tumor models in mice. [4,6,12,13] eEF2K is a critical driver and important therapeutic target in tumor formation and progression of highly aggressive solid cancers. In vitro and in vivo studies show that eEF2K is involved in the increased activity of PI3K/Akt, mTOR, Src/FAK and IGFR and the expression of pathways, cyclin D1 and c-myc, and also modulates the tumor microenvironment by promoting accumulation. eEF2K not only induces oncogenic signaling but also creates an immunosuppressive microenvironment that promotes tumor. [4,6,12]

Overall, recent studies suggest that eEF2K is a critical driver in tumor formation and progression of highly aggressive solid cancers and may serve as an excellent therapeutic target. [14]

Compared to conventional chemotherapy drugs, small molecule kinase inhibitors are used in cancer treatments as targeted therapeutics due to their efficacy and safety. Since the approval of the first tyrosine kinase inhibitor imatinib (STI-571) by the US Food and Drug Administration (FDA) in 2001, many small molecule inhibitors have been developed for cancer treatment. [15] eEF2K cannot be inhibited by known kinase inhibitors. [16]

Due to the clinical importance of eEF2K and its potential as a molecular target for precision medicine, there has recently been great interest in the development of highly effective inhibitors for eEF2K for clinical translation. [17-24]

However, the development of effective inhibitors has been difficult due to the lack of data on the 3D crystal structure of eEF2K. eEF2K cannot be inhibited by well-known pan kinase inhibitors such as staurosporine. [16]

To overcome this challenge, homology modelling based on the similarity of the kinase domain of eEF2K to its close relatives in the alpha kinase family, such as myosin heavy chain kinase 2 (MHCK-2), has recently been developed and used. [17-19]

There has been a focus on the identification of eEF2K inhibitors with various core structures, such as indoles, which are widely used in drug discovery studies due to their effective pharmacological properties. [25]

Indole-containing drugs such as indole alkaloids vincristine and vinblastine are used in the treatment of cancers such as breast cancer and other cancers. [26,27]

Furthermore, indole alkaloids including vallesiachotamine and iso-vallesiachotamine isolated from natural sources and other indoles have been reported to have anticancer activity. [26,28-31]

Studies have shown that some indole derivatives inhibit NFκB and mTOR/PI3K/Akt pathways and these compounds have anticancer activities as well as various biological activities such as anti-HIV and antimycobacterial. [29-32] Etodolac (R,S)-2-[1,8-diethyl-1,3,4-tetrahydropyrano [3,4-b]indol-1-yl]acetic acid containing a pyrano indole ring is an FDA approved nonsteroidal anti-inflammatory drug (NSAID). The S-enantiomer of the drug, present in a racemic mixture, shows selective COX-2 inhibition and is responsible for the biochemical and pharmacological activities. [33,34]

The antineoplastic effects of the etodolac R-enantiomer have been demonstrated in a variety of diseases, including chronic lymphocytic leukemia (CLL), multiple myeloma, colon and prostate cancer cells. [35-38]

Although several small molecules have been reported as eEF2K inhibitors, they are either not specific, i.e. they inhibit many other molecules and kinases, or they do not act on EF2kinase at low concentrations. Therefore, the field for the discovery of effective molecules is open. For example, rottlerin inhibits various other protein kinases at concentrations lower than those required for inhibition of eEF2K. [39]

NH125, an imidazole derivative, was published as an eEF2K inhibitor [39]; however, subsequent studies revealed that this molecule does not inhibit EF2K but rather increases eEF2 phosphorylation. [40-42]

The thiopyran-dicarbonitrile analogue 484954 has been reported as an eEF2K inhibitor. However, it showed inhibition effect in cancer cells at very high doses such as 75 micromolar, which is very weak in cancer cells. TX1918 is not specific as it showed an inhibitory activity against five different kinases. Although compound DFTD was described as an eEF2K inhibitor, the compound was reported to be a reversible covalent inhibitor of eEF2K.

In recent reported studies, according to the drug repositioning study, some approved drugs such as pemetrexed, entecavir, calcium levofolinate and fosbretabulin were proposed as potential EF2K inhibitors and mitoxantrone, which binds directly to EF2K, was identified as a new EF2K inhibitor.

Small molecule kinase inhibitors are used in cancer treatments as targeted therapeutics due to their efficacy and safety. However, eEF2K cannot be inhibited by known kinase inhibitors.

Due to the clinical importance of eEF2K and its potential as a molecular target for precision medicine, there has recently been great interest in the development of highly effective inhibitors for eEF2K for clinical translation.

WO2023001045A1 discloses external anti-inflammatory compound drugs and methods of preparation.

US2010292231A1 discloses indazole compounds for the treatment of inflammatory disorders, demyelinating disorders and cancers.

When the studies available in the art were examined, it was necessary to develop the inventive etodolac derivative compound that can be used in the treatment of cancer and other diseases by developing small molecules as eukaryotic elongation factor 2 kinase (eEF2K) enzyme inhibitors which are effective in breast, pancreatic, brain, ovarian, lung, skin and blood cancers.

### SUMMARY

The object of the present invention is to develop an etodolac derivative compound that can be used in the treatment of cancer and other diseases by developing small molecules as eukaryotic elongation factor 2 kinase (eEF2K) enzyme inhibitors that are effective in breast, pancreatic, brain, ovarian, lung, skin and blood cancers.

Another object of the present invention is to develop an etodolac derivative compound that can be used as a highly potent eEF2K inhibitor at low concentrations using a novel core structure in cancer cells.

### BRIEF DESCRIPTION OF THE DRAWINGS

The compound developed to achieve the object of the present invention are shown in the attached figures.

FIG. 1: 3D and 2D ligand interaction diagram of the inventive compound in the binding site of eEF2K.

FIG. 2: Western blot analysis of the compound of the invention in TNBC cells.

FIG. 3: Western blot analysis for the starting substance etodolac (EC) in TNBC cells.

FIG. 4: FTIR spectrum of the inventive compound.

FIG. 5: ¹HNMR spectrum of the inventive compound.

FIG. 6: ¹³CNMR spectrum of the inventive compound.

FIG. 7: LC-MS/MS spectrum of the inventive compound.

FIG. 8: HPLC chromatogram of the inventive compound.

FIG. 9: Homology model (grey) used in the invention.

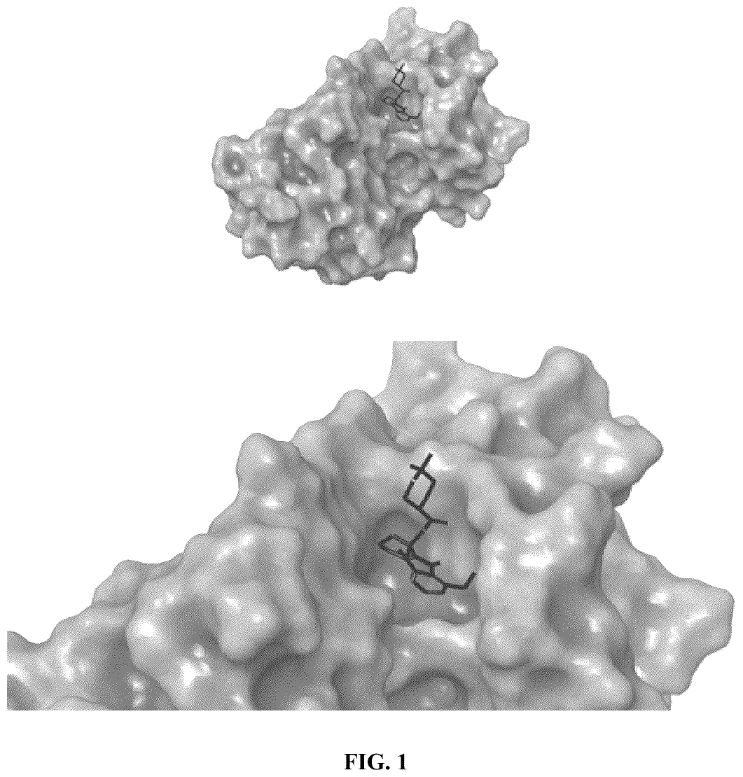

### DETAILED DESCRIPTION OF THE EMBODIMENTS

Within the scope of the invention, the inventive compound with etodolac derivative indole skeleton for EF2K inhibition was designed and synthesized. EF2K inhibitory effect was determined in aggressive triple negative breast cancer cell line (MDA-MB-231).

### A Compound of Formula (A),

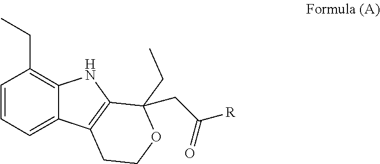

### Characterized by, Wherein R is 4-Methylpiperazine (I)

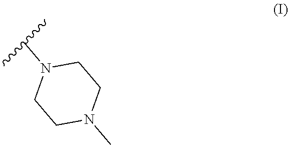 [SMILES: *N1CCN(C)CC1]

The open structure of the inventive compound is given in Formula B. (2-(1,8-diethyl-1,3,4,9-tetrahydropyrano [3,4-b]indol-1-yl)-1-(4-methylpiperazin-1-yl) ethan-1-one)

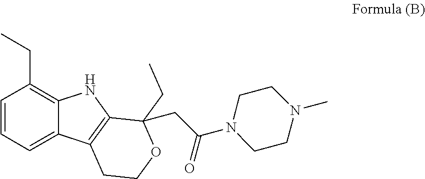 [SMILES: CCc1cccc2c3c([nH]c12)C(CC)(CC(=O)N1CCN(C)CC1)OCC3]

In the process of synthesizing the inventive compound;

Thin layer chromatography (TLC) on silica gel plates (0.25 mm, 60G F254) was used to monitor the reactions. Silica gel (70-230 Mesh, Merck) was used for purification by column chromatography. An X-4 melting point apparatus was used to determine melting points. CAMAG UV light (254 and 365 nm) was used to determine the stains. Fourier transform infrared (FT-IR) spectra and nuclear magnetic resonance (NMR) spectra (¹H NMR and ¹³C NMR, Agilent 600 MHz and JEOL 400 MHz) were recorded for structural analysis of the synthesized compound. [17,19] Shimadzu Scientific Instruments LC-MS/MS 8040 liquid chromatography (LC)-tandem mass spectrometer was used for mass analysis of the synthesized compound. The chemical shift (δ) values in parts per million (ppm) and the coupling constant (J value) expressed in Hertz (Hz) and peaks (δ) in broad, t (single), i (double), u (triple), c (multiple), ii are indicated. High-performance liquid chromatography (HPLC) analysis was performed using a Shimadzu Prominence LC-20A Semi-Preparative HPLC system with a PDA detector on a Shim-pack ODS (H) 250×4.6 mm, 5 μm C18 column.

MeOH: distilled water by volume (%) (70:30) was used as the mobile phase for HPLC analysis for the inventive compound and the flow rate was set to 1 mL min⁻¹. Triple negative breast cancer (TNBC) cell lines (ER-, PR- and HER2-) MDA-MB-231 cells were purchased from the American Type Culture Collection (ATCC) (Manassas, VA, USA). MDA-MB-231 cells were cultured in Dulbecco's modified Eagle's medium (DMEM)/F12 supplemented with 10% FBS (Fetal Bovine Serum) and 100 U mL⁻¹ penicillin and streptomycin. Specific antibodies (p-EF2 (Thr56) (2331S), eEF2 (2332S), eEF2K (3692S) and GAPDH (5174S)) were purchased from Cell Signaling Technology (CST).

### Synthesis of the Inventive Compound

The compound was synthesized using a general method. For this purpose, the coupling reagent EDCI (N-ethyl-N′-(3-dimethylaminopropyl) carbodiimide hydrochloride) was used in the amidation reaction. The reaction was carried out in dimethylformamide (DMF). N-Ethyl-di-isopropyl amine (DIPEA) was used as base. The reaction was successfully carried out at room temperature for 48 h in high yield (Scheme 1).

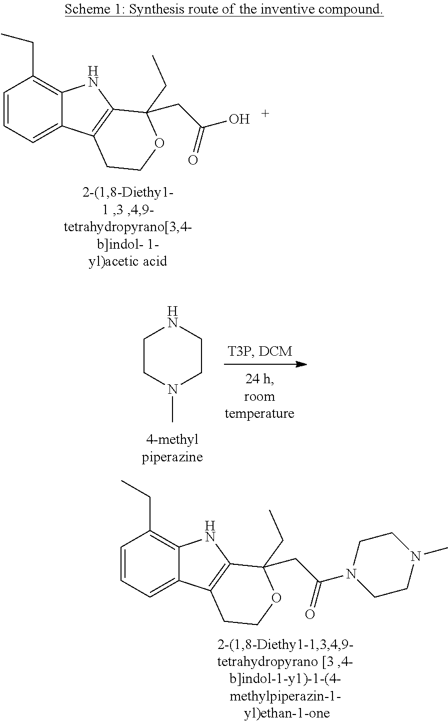

The data obtained for the inventive compound 2-(1,8-Diethyl-1,3,4,9-tetrahydropyrano [3,4-b]indol-1-yl)-1-(4-methylpiperazin-1-yl) ethan-1-one are given below.

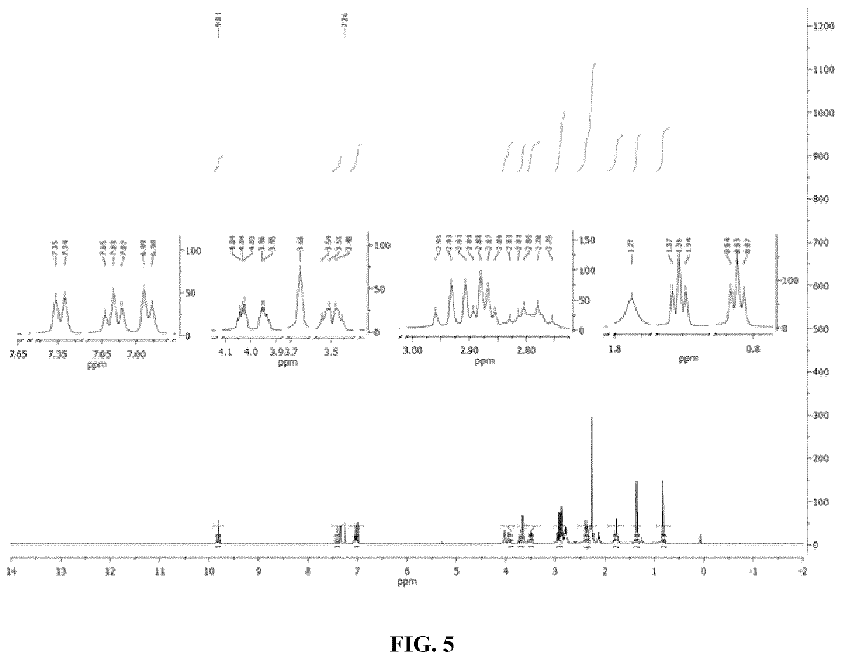

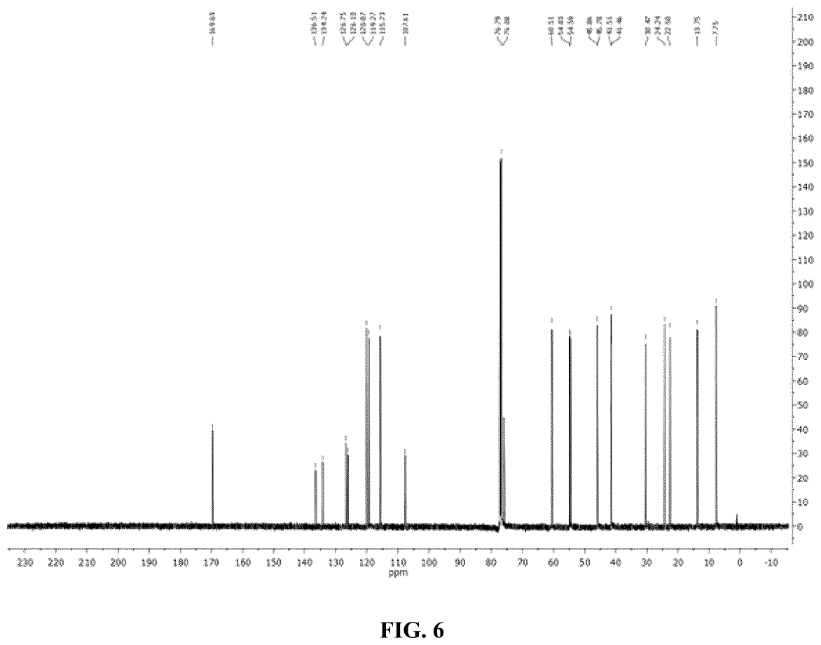

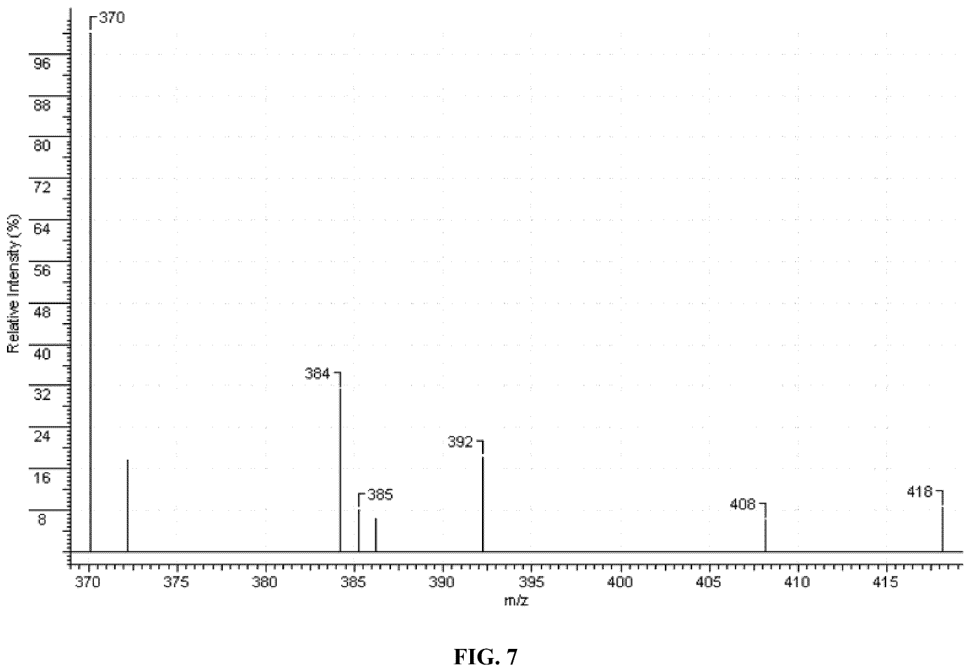

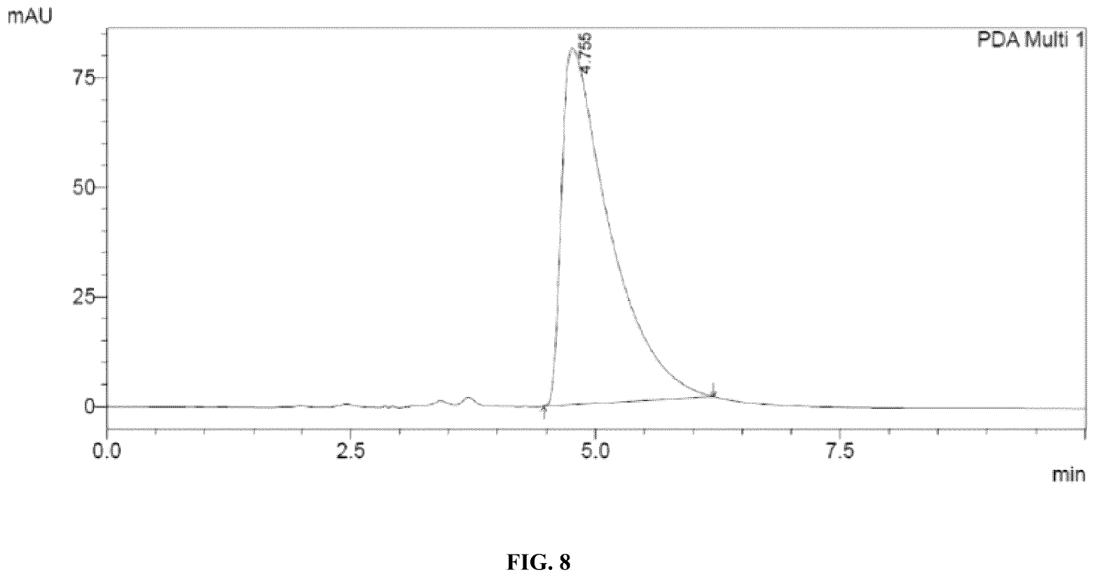

The compound is white. Yield: 55%; Melting point 115-118° C. FT-IR (Vₘₐₓ, cm⁻¹): 3332 (indole —NH), 3051, 2964-2875, 1629 (C═O), 1608. ¹H NMR (600 MHz, CDCl₃) δ H 9.81 (s, 1H, NH), 7.35 (d, 1H, J=7.7 Hz, ArH), 7.03 (t, 1H, J=7.4 Hz, ArH), 6.98 (i, 1H, J=6.9 Hz, ArH), 3.95-4.04 (m, 2H), 3.66 (s, 2H), 3.48-3.54 (m, 2H), 2.75-2.96 (m, 4H), 2.39 (broad, 2H), 2.24-2.32 (m, 4H), 2.11-2.15 (m, 2H), 1.77 (s, 3H, CH3), 1.36 (t, 3H, J=7.6 Hz, CH3), 0.83 (t, 3H, J=7.3 Hz, CH3). ¹³C NMR (150 MHz, CDCl₃) 169.69, 136.51, 134.24, 126.75, 126.10, 120.07, 119.27, 115.73, 107.61, 76.08, 60.51, 54.83, 54.59, 45.86, 45.78, 41.51, 41.46, 30.47, 24.24, 22.50, 13.75, 7.75. LC-MS/MS (ESI) [M+H]+=370. MA. 369 g mol⁻¹. HPLC purity 100%. Formula C₂₂H₃₁N₃O₂. (FIGS. 4-8)

The inventive pyranoindole analogue compound was synthesized using etodolac as starting compound to obtain a secondary aliphatic amide as shown in Scheme 1. The inventive compound was synthesized by amidation reaction between the starting compound and piperidine and heterocyclic amine using a coupling reagent (propane phosphonic acid anhydride (50% solution in ethyl acetate)). Thin layer chromatography (SLC) was used to observe the progress of the reaction. The reactions were carried out in high yields. According to spectroscopic analysis, Fourier transform infrared spectroscopy (FT-IR) showed characteristic peaks of functional groups. Characterization analyses were performed by ¹H NMR and ¹³C NMR. The molecular weight of the compounds was determined by LC-MS/MS analysis. NMR and MS spectra were given in electrospray ionization (ESI). In FT-IR analysis, the amide N—H stretching and C═O amide peaks were obtained at approximately 3300 and 1640 cm⁻¹. The peak for carboxylic acid was observed at 1740 cm⁻¹. The conversion to the amide compound was determined by NMR analysis (FIGS. 4-8).

### Preparation of Protein and Ligand Structure

The 2D structure of the synthesized molecule was drawn, prepared and converted into three-dimensional structures with the help of the Maestro LigPrep module at neutral pH using default settings. Since the crystal structure of eEF2K is not yet known, the alphafold (v.2) UniProt (AF-000418-F1-model_v2) model structure was used. However, the three-dimensional model obtained using alphafold had unrealistic regions, especially in the loop regions of the target protein. It was observed that there was a very good alignment between the 3D model previously developed by the inventors and the alphafold model in the ligand binding sites (residue numbers 107 to 326). Therefore, it was decided to focus on the ligand binding region (region from 107 to 326), since there are particularly unrealistic sections in the loop regions of the target protein obtained using alphafold and there is very good alignment between the alphafold model and the 3D model developed in previous studies in this region with residue numbers from 107 to 326. 200 models were created and their total energies were calculated. Rosetta was used for this. Rosetta is a powerful molecular modelling software package used in the field of computational biology. Protein structures play an important role in understanding protein design, protein docking, protein-DNA interactions and protein-protein interactions. The software includes a set of algorithms that enable computational modelling and analysis of protein structures, leading to significant scientific advances in the field. Rosetta software package applications cover areas such as de novo protein design, enzyme design, ligand docking and structure prediction of biological macromolecules and complexes. Rosetta's algorithms are designed to efficiently explore conformational and sequence space in the search for optimal protein structures and sequences. This capability is invaluable in protein design and optimization where finding the optimal conformation or sequence is critical. The model with the lowest total energy was then validated with Verify3D and PROCHECK servers. The results showed that 88.60% of the residues had average 3D-1D scores of 0.2 or higher.

Docking of the Inventive Compound Ligand to the eEF2K Target

Docking operations were performed with three different docking algorithms. These are Glide extra precision (XP) docking, Glide standard precision (SP) docking and quantum polarized ligand docking (QPLD) algorithms.

Molecular Dynamics (MD) Simulations and Generalised Born and surface area solving (MM/GBSA)

MD simulations were performed to observe the conformational stability of the complex. Desmond program was used for this. The best poses of the complexes were prepared in orthorhombic simple point charge (SPC) water medium. The systems were neutralized with 0.15 M Na and Cl ions. A Nose Hoover thermostat was used to maintain the temperature at 310 K. MD simulations were set up using 1.01 bar pressure (Martyna-Tobias-Klein barostat) conditions. During the simulations 4000 trajectory frames were generated and 200 frames were saved. The highest scoring complexes were further analyzed in terms of binding free energy by MM/GBSA analysis in the Schrodinger Prime module.

### Binary Quantitative Structure Effectiveness Relationship (QSAR) Models

The toxicity prediction analysis of the compound was performed in binary QSAR models of MetaCore/MetaDrug, a comprehensive systems biology analysis package from Clarivate Analytics. The results of the analysis are shown in table 1.

TABLE 1

| | Data of the |
|---|---|
| Name | synthesized compound |
| AMES (TP) (1) | 0.38 |
| Anemia (TP) (2) | 0.21 |
| Carcinogenic Potential (TP) (3) | 0.05 |
| Carcinogenic Potential Female Mice | 0.12 |
| (TP) (4) | |
| Carcinogenic Potential Male Mice (TP) | 0.14 |
| (5) | |
| Carcinogenic Potential Female Rat (TP) | 0.19 |
| (6) | |
| Carcinogenic Potential Male Rat (TP) | 0.16 |
| (7) | |
| Cardiotoxicity (TP) (8) | 0.3 |
| Cytotoxicity model, −log GI50 (M) (TP) | 5.44 |
| (9) | |
| Epididymal toxicity (TP) (10) | 0.19 |
| Genotoxicity (TP) (11) | 0.38 |
| Hepatotoxicity (TP) (12) | 0.15 |
| Kidney necrosis (TP) (13) | 0.07 |
| Kidney weight gain (TP) (14) | 0.06 |
| Liver cholestasis (TP) (15) | 0.43 |
| Liver lipid accumulation (TP) (16) | 0.13 |
| Liver necrosis (TP) (17) | 0.16 |
| Liver weight gain (TP) (18) | 0.18 |
| MRTD (TP) (19) | 0.05 |
| Nasal pathology (TP) (20) | 0.35 |
| Nephron damage (TP) (21) | 0.23 |
| Nephrotoxicity (TP) (22) | 0.06 |
| Neurotoxicity (TP) (23) | 0.35 |
| Pulmonary toxicity (TP) (24) | 0.14 |
| Skin sensitivity (TP) (25) | 44.71 |
| Testicular toxicity (TP) (26) | 0.17 |

Explanations of the names mentioned in Table 1 are given below.
1. Potentially mutagenic (AMES positive), ranging from 0 to 1. A value of 1 is considered AMES positive (mutagenic) and 0 is considered AMES negative (non-mutagenic). The limit value is 0.5. Values close to zero are preferred. The AMES test is based on the reversion of mutations in the histidine operon in the bacterium Salmonella enterica sv Typhimurium.
2. Potential to cause anemia. The limit value is 0.5. Values higher than 0.5 indicate potentially toxic compounds. The training set consists of chemicals and drugs that cause anemia in vivo. Model organisms: human. Model description: Training set N=324, Test set N=51, Sensitivity=0.82, Specificity=0.90, Accuracy=0.86, MCC=0.72.
3. Carcinogenic potential in rats and mice. The limit value is 0.5. Values higher than 0.5 indicate potentially toxic compounds. The training set consists of chemicals and drugs that are carcinogenic in vivo. Model organisms mouse, rat. Model description: Training set N=1210, Test set N=185, Sensitivity=0.96, Specificity=0.90, Accuracy=0.93, MCC=0.86.
4. Carcinogenic potential in female mice. The limit value is 0.5. Values higher than 0.5 indicate potentially toxic compounds. The training set consists of chemicals and drugs that are carcinogenic in vivo. Model organisms: female mouse. Model description: Training set N=640, Test set N=94, Sensitivity=0.90, Specificity=0.93, Accuracy=0.92, MCC=0.83.
5. Carcinogenic potential in male mice. The limit value is 0.5. Values higher than 0.5 indicate potentially toxic compounds. The training set consists of chemicals and drugs that are carcinogenic in vivo. Model organisms Male mouse. Model description: Training set N=584, Test set N=93, Sensitivity=0.91, Specificity=0.
6. Potential for carcinogenic effects in female rats. The limit point is 0.5. Values higher than 0.5 indicate potentially toxic compounds. The training set consists of chemicals and drugs that cause carcinogenic effects in vivo. Model organisms: female rat. Model description: Training set N=667, Test set N=120, Sensitivity=0.90, Specificity=0.96, Accuracy=0.93, MCC=0.86.
7. Potential for carcinogenic effects in male rats. The limit point is 0.5. Values higher than 0.5 indicate potentially toxic compounds. The training set consists of chemicals and drugs that cause carcinogenic effects in vivo. Model organisms: male rat. Model description: Training set N=715, Test set N=117, Sensitivity=0.92, Specificity=0.88, Accuracy=0.90, MCC=0.79.
8. Potential for cardiotoxicity. The limit point is 0.5. Values higher than 0.5 indicate potentially toxic compounds. The training set consists of chemicals and drugs that cause cardiotoxicity in vivo. Model organisms: mouse, rat, human. Model description: Training set N=143, Test set N=30, Sensitivity=0.80, Specificity=1.00, Accuracy=0.90, MCC=0.82.
9. Growth inhibition in MCF7 cell line (Caucasian human breast adenocarcinoma), pGI50. The cut-off point is 6. Values between 6 and 8 correspond to a toxic metabolite, values less than 6 are preferred, values less than 3 are less toxic. Model description: N=1474, R2=0.9, RMSE=0.05.
10. Potential to cause epididymal toxicity. The training set consists of chemicals and drugs that cause epididymal toxicity in vivo. Model organisms: mouse, rat, human. The cut-off point is 0.5. Values higher than 0.5 indicate potentially toxic compounds.
11. Potential to induce genotoxicity. The limit value is 0.5. Values higher than 0.5 indicate potentially toxic compounds. The training set consists of chemicals and drugs that cause genotoxicity in vivo. Model organisms mouse, rat. Model description: Training set N=372, Test set N=86, Sensitivity=0.75, Specificity=0.84, Accuracy=0.79, MCC=0.59.
12. Potential to induce hepatotoxicity. The limit value is 0.5. Values higher than 0.5 indicate potentially toxic compounds. The training set consists of chemicals and drugs that cause hepatotoxicity in vivo. Model organisms: mouse, rat, human. Model description: Training set N=1380, Test set N=231, Sensitivity=0.73, Specificity=0.88, Accuracy=0.81, MCC=0.62.
13. Potential to induce renal necrosis. The limit value is 0.5. Values higher than 0.5 indicate potentially toxic compounds. The training set consists of chemicals and drugs that cause renal necrosis in vivo. Model organisms: mouse, rat, human. Model description: Training set N=221, Test set N=42, Sensitivity=0.96, Specificity=1.00, Accuracy=0.98, MCC=0.95.
14. Potential to induce kidney weight gain. The limit value is 0.5. Values higher than 0.5 indicate potentially toxic compounds. The training set consists of chemicals and drugs that induce kidney weight gain in vivo. Model organisms mouse, rat. Model description: Training set N=240, Test set N=49, Sensitivity=0.95, Specificity=1.00, Accuracy=0.98, MCC=0.96.
15. Potential to cause liver cholestasis. The limit value is 0.5. Values higher than 0.5 indicate potentially toxic compounds. The training set consists of chemicals and drugs that cause cholestasis in vivo. Model organisms: mouse, rat, human. Model description: Training set N=218, Test set N=35, Sensitivity=0.79, Specificity=0.67, Accuracy=0.74, MCC=0.46.
16. Potential to cause lipid accumulation in the liver. The limit value is 0.5. Values higher than 0.5 indicate potentially toxic compounds. The training set consists of chemicals and drugs that cause lipid accumulation in vivo. Model organisms: mouse, rat, human. Model description: Training set N=172, Test set N=28, Sensitivity=0.80, Specificity=0.85, Accuracy=0.82, MCC=0.64.
17. Potential for liver necrosis. The limit value is 0.5. Values higher than 0.5 indicate potentially toxic compounds. The training set consists of chemicals and drugs that induce hepatic necrosis in vivo. Model organisms: mouse, rat, human. Model description: Training set N=300, Test set N=57, Sensitivity=0.91, Specificity=0.91, Accuracy=0.91, MCC=0.82.
18. Potential to increase liver weight. The limit value is 0.5. Values higher than 0.5 indicate compounds that potentially alter liver weight. The training set consists of chemicals and drugs that cause liver weight gain in vivo. Model organisms mouse, rat. Model description: Training set N=292, Test set N=52, Sensitivity=1.00, Specificity=1.00, Accuracy=1.00, MCC=1.00.
19. Maximum Recommended Therapeutic Dose, log mg/kg bm/day, range −5 to 3. The limit point is 0.5. Chemicals with a high log MRTD can be classified as slightly toxic compounds, while chemicals with a low log MRTD can be classified as highly toxic compounds. Model description: N=1209, R2=0.86, RMSE=0.42.
20. Potential to induce nasal pathology. The training set consists of chemicals and drugs that induce nasal pathology in vivo. Model organisms: mouse, rat, human. The cut-off point is 0.5. Values higher than 0.5 indicate potentially toxic compounds. Model description: Training set N=246, Test set N=47, Sensitivity=1.00, Specificity=0.93, Accuracy=0.96, MCC=0.92.
21. Potential to cause nephron damage. Cut-off value is 0.5. Values higher than 0.5 indicate potentially toxic compounds. The training set includes chemicals and drugs that cause nephron damage in vivo. Model organisms: mouse, rat, human. Model description: Training set N=598, Test set N=109, Sensitivity=0.91, Specificity=1.00, Accuracy=0.96, MCC=0.93.
22. Potential to cause nephrotoxicity. Cut-off value is 0.5. Values higher than 0.5 indicate potentially toxic compounds. The training set includes chemicals and drugs that cause nephrotoxicity in vivo. Model organisms: mouse, rat, human. Model description: Training set N=847, Test set N=154, Sensitivity=0.90, Specificity=0.84, Accuracy=0.87, MCC=0.74.
23. Potential to cause neurotoxicity. The training set includes chemicals and drugs that cause neurotoxicity in vivo. Model organisms: mouse, rat, human. Cut-off value is 0.5. Values higher than 0.5 indicate potentially toxic compounds. Model description: Training set N=175, Test set N=34, Sensitivity=0.94, Specificity=0.94, Accuracy=0.94, MCC=0.88.
24. Potential to cause pulmonary toxicity. The training set includes chemicals and drugs that cause pulmonary toxicity in vivo. Model organisms: mouse, rat, human. Cut-off value is 0.5. Values higher than 0.5 indicate potentially toxic compounds. Model description: Training set N=482, Test set N=87, Sensitivity=0.89, Specificity=0.88, Accuracy=0.89, MCC=0.77.
25. Skin sensitization potential, effective concentration 3, expressed in %. Values higher than 10% indicate weak and moderate sensitizers. Model description: N=89, R2=0.67, RMSE=22.56.
26. Consists of chemicals and drugs that cause testicular toxicity in vivo. Model organisms: mouse, rat, human. The cut-off point is 0.5. Values higher than 0.5 indicate potentially toxic compounds. Model description: Training set N=439, Test set N=88, Sensitivity=0.81, Specificity=0.85, Accuracy=0.83, MCC=0.66. a. Potential activity against cancer. The cut-off point is 0.5. Values higher than 0.5 indicate potentially active compounds. The training set consists of approved drugs. Model description: Training set N=886, Test set N=167, Sensitivity=0.89, Specificity=0.83, Accuracy=0.86, MCC=0.72.
   Investigation of the Interactions of the Inventive Compound Ligand with eEF2K Target

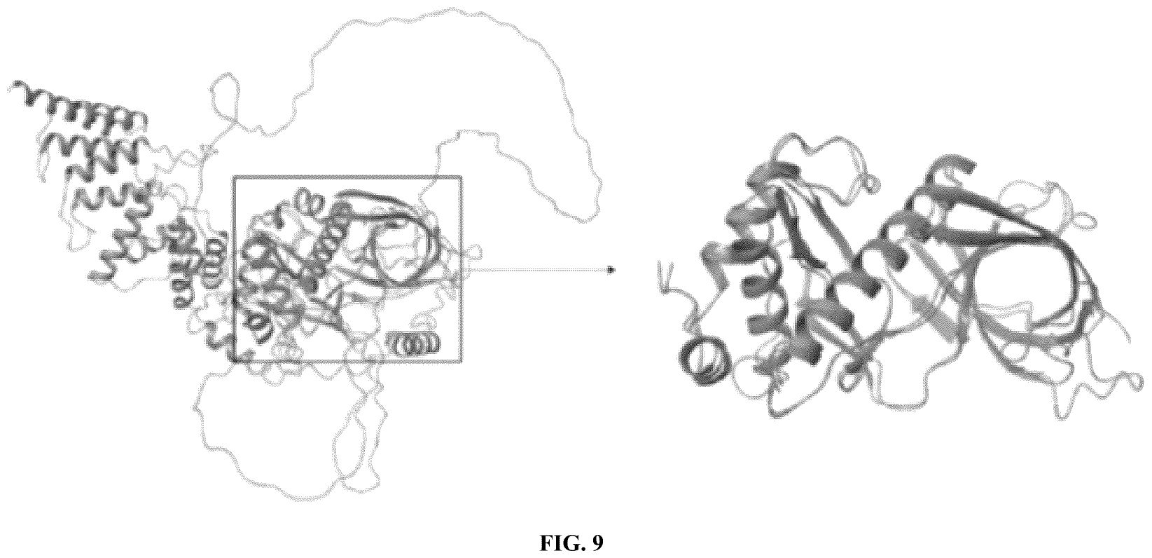

Three different binding algorithms (Glide/SP (standard precision), Glide/XP (extra precision) and quantum-polarized ligand binding (QPLD)) were used to better understand the interaction mechanism of the synthesized compound in the eEF2K binding site. So far, the eEF2K crystal structure has not been analysed. However, 3D structure prediction using alphafold (v.2) is available in UniProt (AF000418-F1-model_v2). When we superimposed our 3D model with the alphafold model, both models aligned with high similarity in the ligand binding site (residue numbers 107 to 326 region). The root mean square deviation (RMSD) between the two models was found to be 1.5 Å. (FIG. 9)

It was decided to focus on the ligand binding site (region 107 to 326), as there were sections of the loop regions of the target protein obtained using alphafold that were not particularly realistic and there was very good alignment between the alphafold model and the 3D model developed in previous studies in the region with residue numbers 107 to 326. The region with residue numbers 107 to 326 was extracted from the alphafold model and this model was used as an input structure in the Rosetta rest. “Relax” Rosetta (https://www.rosettacommons.org/) is the main protocol for optimizing all atoms in the force field and searches the local conformational space around the starting structure. Rosetta's all-atom total energy scores are used to evaluate the different conformations obtained. In this context, 200 models were created and their total energies were calculated using all-atom total energy scores with Rosetta. Then, the model with the lowest total energy (model #41) was verified on Verify3D (https://www.doe-mbi.ucla.edu/verify3d/) and PROCHECK (https://www.ebi.ac.uk/thornton-srv/software/PROCHECK/) servers.

The results showed that 88.60% of the residues had average 3D-1D scores of 0.2 or higher. After protein preparation, this model was used in different molecular docking studies (Glide/SP, Glide/XP and QPLD). The best docking poses were used in all-atom molecular dynamics (MD) simulations. When MD simulations were initiated with Glide/SP and Glide/XP, the ligand was not very stable in the binding site and left the active site during the simulation. However, when the simulations were initiated with the best docking poses of QPLD, the ligand interactions remained stable throughout the MD simulations.

Table 2 shows the docking score and the average molecular mechanics/generalized Born surface area calculations (MM/GBSA) score of the studied compound.

TABLE 2

| | Glide/SP | | Glide/XP | | QPLD | |
|---|---|---|---|---|---|---|
| | Docking | Average | Docking | | Docking | Average |
| | score | MM/GBSA | score | | score | MM/GBSA |
| Synthesized | (kcal | (kcal | (kcal | Synthesized | (kcal | (kcal |
| compound | mol⁻¹) | mol⁻¹) | mol⁻¹) | compound | mol⁻¹) | mol⁻¹) |
| Inventive | −3.8 | NA | −2.6 | NA | −3.6 | −36.8 |
| compound | | | | | | |

Etodolac docking score (Glide/SP) was measured as −5.1 kcal mol⁻¹. MD simulations for the synthesized compound were performed using the Desmond MD simulation program and mean binding free energy calculations were performed for all complexes using the MM/GBSA approach. The trajectories of these MD simulations were analyzed as the docking poses showed a better stability in the binding site when the simulations were initiated with QPLD. In conclusion, the determination of the correct protonation state of the ligand, which takes into account the charge polarization induced by the residues in the binding site, is critical for binding. Using ab initio charge calculations that overcome this limitation, QPLD was subjected to 200 ns MD simulation using the resulting complexes as starting structures. Ligand RMSDs aligned to LigFitProt and LigFitLig were checked. The toxicity profiles of this compound were also checked using Clarivate Analytic's MetaCore/MetaDrug program. The results showed that the compound did not have serious toxicity issues.

eEF2K Activity

Western blot analysis was carried out according to previously reported studies to evaluate the eEF2K inhibition activity for the synthesized compounds. The synthesized compound was applied to MDA-MB-231 cells in the range of various concentrations from 2.5 to 20 micromolar for 2 h treatment. The expression levels of p-EF2 (Thr56) inhibition and eEF2K activity were determined by using related antibodies such as p-EF2 (Thr56), eEF2 and eEF2K. Glyceraldehyde 3-phosphate dehydrogenase (GAPDH) was used as a loading control.

### Biological Assessment

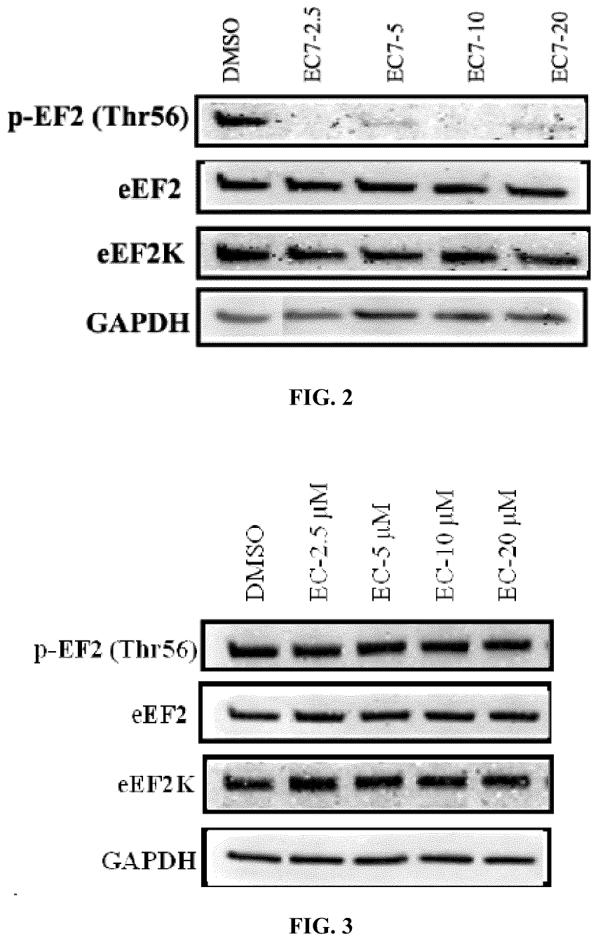

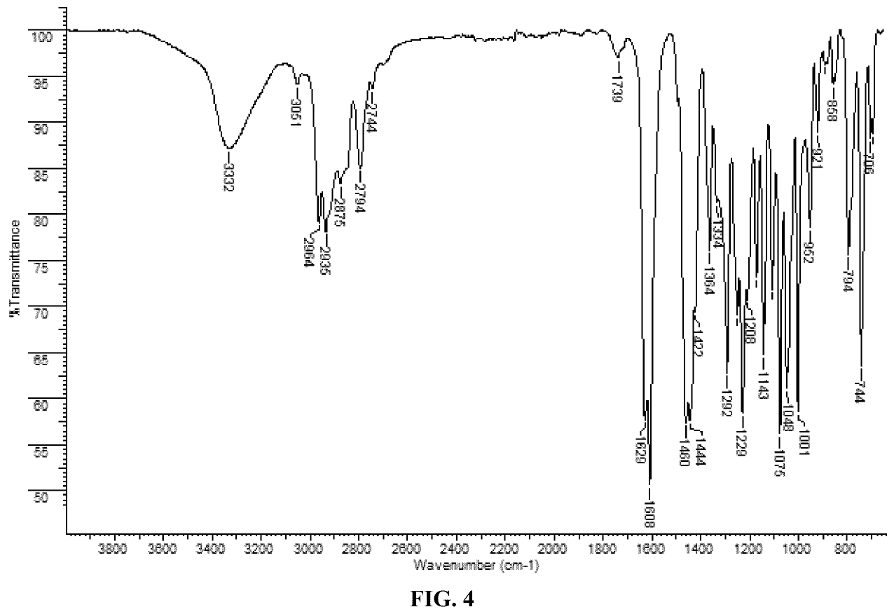

To determine the effects of the synthesized compounds in inhibiting eEF2K activity in TNBC, MDA-MB-231 cells, we performed western blot analysis at concentrations ranging from 2.5 micromolar to 20 micromolar in a dose-dependent manner. eEF2K inhibition was detected by decreased phosphorylation levels of EF2 (p-EF2-Thr-56), a direct downstream substrate of the enzyme. Compared to the original drug etodolac (EC), a marked inhibition of eEF2K was observed with the compound of the invention, with reduced levels of p-EF2 at 2.5 micromolar (FIGS. 2-3).

The inventive compound having an ethylene linker between a heterocyclic amine ring and pyrano [3,4-b]indole exhibited high inhibition potential. Overall, it exhibited highly potent activity in TNBC cells (FIG. 4).

On the basis of the literature, previously reported eEF2K inhibitors (A484954, TX1918) did not show significant eEF2K inhibition. For example, A484954 and TX1918 were reported to inhibit eEF2K at concentrations as high as 100 micromolar and 10 micromolar, respectively. [44-46] TX1918 has been shown to show non-specific effects and inhibit other kinases. Recently, several compounds containing a coumarin scaffold have been shown to be effective as eEF2K inhibitors for targeting. [17-21]

Therefore, we focused on identifying highly potent eEF2K inhibitors at low concentrations using a novel core structure in cancer cells. The findings showed that amide-substituted pyrano-indole derivatives were highly effective in inhibiting eEF2K and could be used as potent eEF2K-targeted anticancer candidates in tumor models.

### Structure Activity Relationship (SAR)

Based on combined in silico and in vitro studies, the results were evaluated by structure-activity relationship (SAR). The inventive compound showed the highest phospho-eEF2 (Thr56) inhibition in MDA-MB-231 cells at a low concentration of 2.5 micromolar for 2 hours of treatment (FIG. 2).

Etodolac (EC) drug did not show any inhibition potential in MDA-MB-231 cells (FIG. 3).

As a result, the contribution of the structural differences between the synthesized analogue compound and the drug EC to the inhibition of its effect was observed (FIGS. 2-3).

The development of small molecules as eEF2K inhibitors effective in cancer treatment and finding candidate compounds for clinical trials will provide new unique molecules to very expensive cancer drugs.

The drug etodolac, which is used as a COX2 inhibitor, has been synthesized as a new analogue effective in cancer through its structure, making it easy to use in the clinic.

Inhibition of the eEF2K enzyme in cancer cells will also be effective in breast cancer, pancreatic cancer, ovarian cancer, brain tumors, melanoma, lung and prostate cancers and some blood cancers, which are extremely fatal and have no definitive treatment.

Since eEF2K is genetically similar to human eEF2K in other species (homology), it is likely to play an important role in animal tumors and may find a place in veterinary medicine for the treatment of domestic animals (dogs, cats).

It is also possible that eEF2K inhibitors may be used in the above-mentioned diseases of the heart (atherosclerosis), depression and brain degenerative diseases of our age, such as Alzheimer's disease, which are associated with increased activity of EF2K.

The effects of the discovered compounds against other types of cancer and/or other diseases can be investigated and the field of use-treatment can be expanded.

### REFERENCES

1. A. G. Ryazanov, E. A. Shestakova and P. G. Natapov, Nature, 1988, 334(6178), 170-173.
2. A. G. Ryazanov and E. K. Davydova, FEBS Lett., 1989, 251, 187-190.
3. J. W. Kenney, C. E. Moore, X. Wang, C. G. Proud, Adv. Biol. Regul., 2014, 55, 15-27.
4. I. Tekedereli, S. N. Alpay, C. D. Tavares, et al. and B. Ozpolat, PLoS One, 2012, 7, e41171.
5. R. Shahbazi, E. Asik, N. Kahraman, M. Turk, B. Ozpolat, K. Ulubayram, Nanomedicine, 2017, 12(16), 1961-1973.
6. E. Asik, Y. Akpinar, A. Caner, et al. and B. Ozpolat, Nanomedicine, 2019, 14(17), 2315-2338.
7. A. A. Ashour, N. Gurbuz, S. N. Alpay, A. A. Abdel-Aziz, A. M. Mansour, L. Huo, B. Ozpolat, J. Cell. Mol. Med., 2014, 18, 2235-2251.
8. A. A. Ashour, A. A. Abdel-Aziz, A. M. Mansour, S. N. Alpay, L. Huo, B. Ozpolat, Apoptosis, 2014, 19, 241-258.
9. H. A. Bircan, N. Gurbuz, A. Pataer, et al. and B. Ozpolat, Lung Cancer, 2018, 124, 31-39.
10. M. A. Erdogan, A. Ashour, E. Yuca, K. Gorgulu, B. Ozpolat, Cell. Signaling, 2021, 81, 109938.
11. R. Bayraktar, M. Pichler, P. Kanlikilicer, et al. and B. Ozpolat, Oncotarget, 2017, 8(7), 11641-11658.
12. R. Bayraktar, C. Ivan, E. Bayraktar, et al. and B. Ozpolat, Clin. Cancer Res., 2018, 24(17), 4225-4241.
13. A. Gorur, R. Bayraktar, C. Ivan, H. et al. and B. Ozpolat, Mol. Ther.-Nucleic Acids, 2021, 23, 930-943.
14. X. Wang, J. Xie, C. G. Proud, Cancers, 2017, 9(12), 162.
15. L. Zhong, Y. Li, L. Xiong, W. Wang, et al., Signal Transduction Targeted Ther., 2021, 6(1), 201.
16. R. Liu and C. G. Proud, Acta Pharmacol. Sin., 2016, 37, 285-294.
17. F. Comert Onder, S. Durdagi, N. Kahraman, T. N. Uslu, H. Kandemir, E. B. Atici, B. Ozpolat, M. Ay, Bioorg. Chem., 2021, 116, 105296.
18. F. Comert Onder, N. Kahraman, E. Bellur Atici, A. Cagir, H. Kandemir, G. Tatar, T. Taskin Tok, G. Kara, B. Karliga, S. Durdagi, M. Ay, B. Ozpolat, ACS Pharmacol. Transl. Sci., 2021, 4(2), 926-940, Correction. ACS Pharmacol. Transl. Sci., 2021, 4(3), 1247.
19. F. Comert Onder, S. Durdagi, K. Sahin, B. Ozpolat, M. Ay, J. Chem. Inf. Model., 2020, 60(3), 1766-1778.
20. F. Comert Onder, S. Sagbas Suner, N. Sahiner, M. Ay, B. Ozpolat, Pharm. Res., 2020, 37(3), 63.
21. M. Ay, B. Ozpolat, F. Comert Onder, T. Taşkin Tok, E. Bellur Atici, B. Karliga, H. Kandemir, A. Cagir, N. Sahiner, G. Tatar, WO2019/240701A1, 2019.
22. S. Zhu, M. Liao, H. Tan, L. Zhu, et al., J. Med. Chem., 2021, 64(13), 8870-8883.
23. T. Xiao, R. Liu, C. G. Proud, M. W. Wang, Acta Pharm. Sin. B, 2016, 6(6), 557-563.
24. B. Zhang, J. Zou, Q. Zhang, Z. Wang, et al., Int. J. Mol. Sci., 2021, 22(5), 2408.
25. A. M. S. El-Sharief, Y. A. Ammar, A. Belal, et al., Bioorg. Chem., 2019, 85, 399-412.
26. H. Sachdeva, J. Mathur, A. Guleria, J. Chil. Chem. Soc., 2020, 65(3), 4900-4907.
27. E. Martino, G. Casamassima, S. Castiglione, et al., ACS Med. Chem. Lett., 2018, 28(17), 2816-2826.
28. D. P. Mishra, M. A. Khan, D. K. Yadav, et al., ChemistrySelect, 2018, 3, 8468-8472.
29. N. Devi, K. Kaur, A. Biharee, V. Jaitak, Anti-Cancer Agents Med. Chem., 2021, 21(14), 1802-1824.
30. K. Kaur, V. Jaitak, Anti-Cancer Agents Med. Chem., 2019, 19(8), 962-983.
31. J. S. Sidhu, R. Singla, V. Mayank, Anti-Cancer Agents Med. Chem., 2015, 16(2), 160-173.
32. M. G. Laporte, R. W. Jackson, T. L. Draper, et al., ChemMedChem, 2008, 3(10), 1508-1515.
33. C. A. Demerson, L. G. Humber, N. A. Abraham, et al., J. Med. Chem., 1983, 26(12), 1778-1780.
34. P. çikla-Süzgün, N. Kaushik-Basu, A. Basu, et al., J. Enzyme Inhib. Med. Chem., 2015, 30(5), 778-785.
35. J. Behari, G. Zeng, W. Otruba, M. D. Thompson, et al., J. Hepatol., 2007, 46(5), 849-857.
36. S. K. Kolluri, M. Corr, S. Y. James, et al., Proc. Natl. Acad. Sci. U.S.A, 2005, 102, 2525-2530.
37. H. Yasui, T. Hideshima, M. Hamasaki, et al., Blood, 2005, 106, 706-712.
38. P. çila, D. Özsavci, Ö. Bingöl-Özakpinar, et al., Arch. Pharm., 2013, 346(5), 367-379.
39. Gschwendt M, Kittstein W, Marks F. FEBS Lett 1994; 338:85-8.
40. Arora S, Yang J M, Kinzy T G, et al. Cancer Res 2003; 63:6894-9.
41. Chen Z, Gopalakrishnan S M, Bui M H, et al., J Biol Chem 2011; 286:43951-8.
42. Devkota A K, Tavares C D, Warthaka M, et al., Biochemistry 2012; 51:2100-12.
43. Edupuganti, R., Wang, Q., Tavares, C. D. J., et al., Bioorg. Med. Chem. 2014; 22, 4910-4916.
44. Hori, H., Nagasawa, H., Ishibashi, M., et al., Bioorg. Med. Chem. 2002; 10, 3257-3265.
45. Devkota, A. K., Edupuganti, R., Yan, C., et al., Chembiochem. 2014b; 15 (16), 2435-42.
46. Zhu, S., Liao, M., Tan, H., et al., J Med Chem. 2021; 64 (13), 8870-8883.

What is claimed is:

1. A compound of formula (A), 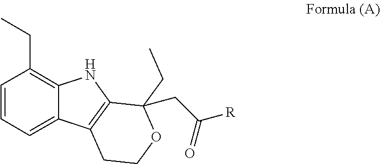
wherein R is 4-methylpiperazine (I), 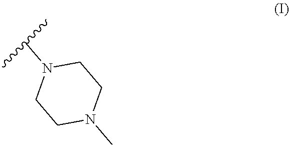 [SMILES: *N1CCN(C)CC1]
